(19) United States
(12) Patent Application Publication
(10) Pub. No.: US 2005/0020832 A1
(43) Pub. Date: Jan. 27, 2005

(54) Pyridone, pyridazone and triazone derivatives as lp-pla2 inhibitors

(76) Inventors: Stephen Christopher Martin Fell, Harlow (GB); Deirdre Mary Bernadette Hickey, Stevenage (GB); Colin Andrew Leach, King of Prussia, PA (US); John Liddle, Stevenage (GB); Ivan Leo Pinto, Stevenage (GB); Stephen Allan Smith, Stevenage (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/495,022

(22) PCT Filed: Nov. 8, 2002
(86) PCT No.: PCT/EP02/12507

(30) Foreign Application Priority Data
Nov. 10, 2001 (GB) 0127143.6

(51) Int. Cl. C07D 237/02; C07D 241/02

(52) U.S. Cl. 544/182; 544/310; 544/406

(57) ABSTRACT

Compounds of the formula (I) are inhibitors of the enzyme Lp-PLA2 and are of use in therapy, in particular for treating atherosclerosis.

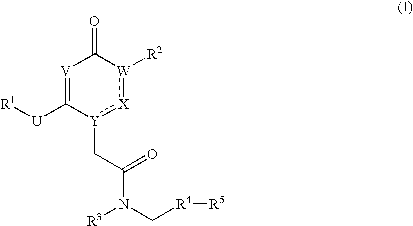

The present invention relates to certain novel pyridone, pyridazone and triazinone compounds, processes for their preparation, intermediates useful in their preparation, pharmaceutical compositions containing them and their use in therapy, in particular in the treatment of atherosclerosis.

WO 95/00649 (SmithKline Beecham plc) describes the phospholipase A₂ enzyme Lipoprotein Associated Phospholipase A₂ (Lp-PLA₂), the sequence, isolation and purification thereof, isolated nucleic acids encoding the enzyme, and recombinant host cells transformed with DNA encoding the enzyme. Suggested therapeutic uses for inhibitors of the enzyme included atherosclerosis, diabetes, rheumatoid arthritis, stroke, myocardial infarction, reperfusion injury and acute and chronic inflammation. A subsequent publication from the same group further describes this enzyme (Tew D et al, Arterioscler Thromb Vas Biol 1996:16;591-9) wherein it is referred to as LDL-PLA₂. A later patent application (WO 95/09921, Icos Corporation) and a related publication in Nature (Tjoelker et al, vol 374, 6 Apr. 1995, 549) describe the enzyme PAF-AH which has essentially the same sequence as Lp-PLA₂ and suggest that it may have potential as a therapeutic protein for regulating pathological inflammatory events.

It has been shown that Lp-PLA₂ is responsible for the conversion of phosphatidylcholine to lysophosphatidylcholine, during the conversion of low density lipoprotein (LDL) to its oxidised form. The enzyme is known to hydrolyse the sn-2 ester of the oxidised phosphatidylcholine to give lysophosphatidylcholine and an oxidatively modified fatty acid. Both products of Lp-PLA₂ action are biologically active with lysophosphatidylcholine in particular having several pro-atherogenic activities ascribed to it, including monocyte chemotaxis and induction of endothelial dysfunction, both of which facilitate monocyte-derived macrophage accumulation within the artery wall. Inhibition of the Lp-PLA₂ enzyme would therefore be expected to stop the build up of these macrophage enriched lesions (by inhibition of the formation of lysophosphatidylcholine and oxidised free fatty acids) and so be useful in the treatment of atherosclerosis.

The increased lysophosphatidylcholine content of oxidatively modified LDL is also thought to be responsible for the endothelial dysfunction observed in patients with atherosclerosis. Inhibitors of Lp-PLA₂ could therefore prove beneficial in the treatment of this phenomenon. An Lp-PLA₂ inhibitor could also find utility in other disease states that exhibit endothelial dysfunction including diabetes, hypertension, angina pectoris and after ischaemia and reperfusion.

In addition, Lp-PLA₂ inhibitors may also have a general application in any disorder that involves activated monocytes, macrophages or lymphocytes, as all of these cell types express Lp-PLA₂. Examples of such disorders include psoriasis.

Furthermore, Lp-PLA₂ inhibitors may also have a general application in any disorder that involves lipid oxidation in conjunction with LpPLA₂ activity to produce the two injurious products, lysophosphatidylcholine and oxidatively modified fatty acids. Such conditions include the aforementioned conditions atherosclerosis, diabetes, rheumatoid arthritis, stroke, myocardial infarction, ischaemia, reperfusion injury and acute and chronic inflammation.

Patent applications WO 96/12963, WO 96/13484, WO96/19451, WO 97/02242, WO97/217675, WO 97/217676, WO 96/41098, and WO 97/41099 (SmithKline Beecham plc) disclose inter alia various series of 4-thionyl/sulfinyl/sulfonyl azetidinone compounds which are inhibitors of the enzyme Lp-PLA₂. These are irreversible, acylating inhibitors (Tew et al, Biochemistry, 37, 10087, 1998).

A further class of compounds has now been identified which are non-acylating inhibitors of the enzyme Lp-PLA₂. Thus, WO 99/24420, WO 00/10980, WO 00/66566, WO 00/66567 and WO 00/68208 (SmithKline Beecham plc) disclose a class of pyrimidone compounds. We have now found that the pyrimidone ring may be replaced by a pyridone, pyridazone or triazinone ring to give compounds having good activity as inhibitors of the enzyme Lp-PLA₂.

Accordingly, the present invention provides a compound of formula (I): 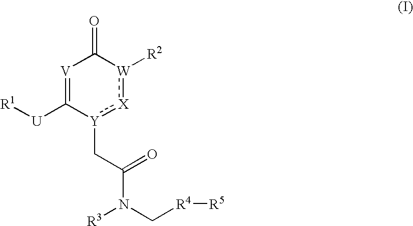
in which:
R¹ is an aryl group, optionally substituted by 1, 2, 3 or 4 substituents which may be the same or different selected from C₍₁₋₆₎alkyl, C₍₁₋₆₎alkoxy, C₍₁₋₆₎alkylthio, hydroxy, halogen, CN, mono to perfluoro-C₍₁₋₄₎alkyl, mono to perfluoro-C₍₁₋₄₎alkoxyaryl, and arylC₍₁₋₄₎alkyl;
when W is C, R² is hydrogen, halogen, C₍₁₋₃₎alkyl, C₍₁₋₃₎alkoxy, hydroxyC₍₁₋₃₎alkyl, C₍₁₋₃₎alkylthio, C₍₁₋₃₎alkylsulphinyl, aminoC₍₁₋₃₎alkyl, mono- or di-C₍₁₋₃₎alkylaminoC₍₁₋₃₎alkyl, C₍₁₋₃₎alkylcarbonylaminoC₍₁₋₃₎alkyl, C₍₁₋₃₎alkoxyC₍₁₋₃₎alkylcarbonylaminoC₍₁₋₃₎alkyl, C₍₁₋₃₎alkylsulphonylaminoC₍₁₋₃₎alkyl, C₍₁₋₃₎alkylcarboxy, or CR⁶R⁷R⁸; or
when W is N, R² is hydrogen, C₍₁₋₃₎alkyl, hydroxyC₍₁₋₃₎alkyl, aminoC₍₁₋₃₎alkyl, mono- or di-C₍₁₋₃₎alkylaminoC₍₁₋₃₎alkyl, C₍₁₋₃₎alkylcarbonylaminoC₍₁₋₃₎alkyl, C₍₁₋₃₎alkoxyC₍₁₋₃₎alkylcarbonylaminoC₍₁₋₃₎alkyl, C₍₁₋₃₎alkylsulphonylaminoC₍₁₋₃₎alkyl, or CR⁶R⁷R⁸;
R³ is hydrogen, C₍₁₋₆₎alkyl which may be unsubstituted or substituted by 1, 2 or 3 substituents selected from hydroxy, halogen, OR⁹, COR⁹, carboxy, COOR⁹, CONR¹⁰R¹¹, NR¹⁰R¹¹, NR⁹COR¹², mono- or di-(hydroxyC₍₁₋₆₎alkyl)amino and N-hydroxyC₍₁₋₆₎alkyl-N—C₍₁₋₆₎alkylamino; or
R³ is Het-C₍₀₋₄₎alkyl in which Het is a 5- to 7-membered heterocyclyl ring comprising and optionally O or S, and in which N may be substituted by COR⁹, COOR⁹, CONR¹⁰R¹¹, or C₍₁₋₆₎alkyl optionally substituted by 1, 2 or 3 substituents selected from hydroxy, halogen, OR⁹, COR⁹, carboxy, COOR⁹, CONR¹⁰R¹¹ or NR¹⁰R¹¹, for instance, piperidin-4-yl, pyrrolidin-3-yl;
R⁴ is an aryl or a heteroaryl ring optionally substituted by 1, 2, 3 or 4 substituents which may be the same or different selected from C₍₁₋₆₎alkyl, C₍₁₋₆₎alkoxy, C₍₁₋₆₎alkylthio, arylC₍₁₋₆₎alkoxy, hydroxy, halogen, CN, COR⁹, carboxy, COOR⁹, NR⁹COR¹², CONR¹⁰R¹¹, SO₂NR¹⁰R¹¹, NR⁹SO₂R¹², NR¹⁰R¹¹, mono to perfluoro-C₍₁₋₄₎alkyl and mono to perfluoro-C₍₁₋₄₎alkoxy;
R⁵ is an aryl or a heteroaryl ring which is further optionally substituted by 1, 2, 3 or 4 substituents which may be the same or different selected from C₍₁₋₁₈₎alkyl, C₍₁₋₁₈₎alkoxy, C₍₁₋₆₎alkylthio, C₍₁₋₆₎alkylsulfonyl, arylC₍₁₋₆₎alkoxy, hydroxy, halogen, CN, COR⁹, carboxy, COOR⁹, CONR¹⁰R¹¹, NR⁹COR¹², SO₂NR¹⁰R¹¹, NR⁹SO₂R¹², NR¹⁰R¹¹, mono to perfluoro-C₍₁₋₄₎alkyl and mono to perfluoroC₍₁₋₄₎alkoxy, or C₍₅₋₁₀₎alkyl;
R⁶ and R⁷ are each hydrogen or C₍₁₋₄₎alkyl, or R⁶ and R⁷ together with the intervening carbon atom form a C₍₃₋₆₎cycloalkyl ring;
R⁸ is an aryl or heteroaryl group, optionally substituted by 1, 2, 3 or 4 substituents which may be the same or different selected from C₍₁₋₁₈₎alkyl, C₍₁₋₁₈₎alkoxy, C₍₁₋₁₈₎alkylthio, arylC₍₁₋₁₈₎alkoxy, hydroxy, halogen, CN, COR⁹, carboxy, COOR⁹, CONR¹⁰R¹¹, NR⁹COR¹², SO₂NR¹⁰R¹¹, NR⁹SO₂R¹², NR¹⁰R¹¹, mono to perfluoro-C₍₁₋₄₎alkyl and mono to perfluoro-C₍₁₋₄₎alkoxy; or
R⁸ is an aryl or heteroaryl group, optionally substituted by 1 substituent selected from CH₂COOH or a salt thereof, CH₂COOR¹³, CH₂CONR¹⁰R¹¹, CH₂CN, (CH₂)ₘNR¹⁰R¹¹, (CH₂)ₘOH and (CH₂)ₘOR⁹ where m is an integer from 1 to 3, optionally in combination with a further substituent selected from C₍₁₋₁₈₎alkyl, C₍₁₋₁₈₎alkoxy, C₍₁₋₁₈₎alkylthio, arylC₍₁₋₁₈₎alkoxy, hydroxy, halogen, CN, COR⁹, carboxy, COOR⁹, CONR¹⁰R¹¹, NR⁹COR¹², SO₂NR¹⁰R¹¹, NR⁹SO₂R¹², NR¹⁰R¹¹, mono to perfluoro-C₍₁₋₄₎alkyl and mono to perfluoro-C₍₁₋₄₎alkoxy;
R⁹ and R¹² are independently hydrogen or C₍₁₋₁₂₎alkyl, for instance C₍₁₋₄₎alkyl (e.g. methyl or ethyl);
R¹⁰ and R¹¹ which may be the same or different is each selected from hydrogen, or C₍₁₋₁₂₎alkyl, or R¹⁰ and R¹¹ together with the nitrogen to which they are attached form a 5- to 7 membered ring optionally containing one or more further heteroatoms selected from oxygen, nitrogen and sulphur, and optionally substituted by one or two substituents selected from hydroxy, oxo, C₍₁₋₄₎alkyl, C₍₁₋₄₎alkylcarboxy, aryl, e.g. phenyl, or aralkyl, e.g benzyl, for instance morpholine or piperazine;
R¹³ is C₍₁₋₄₎alkyl or a pharmaceutically acceptable in vivo hydrolysable ester group;
U is a C₍₂₋₄₎alkylene group optionally substituted by 1, 2 or 3 substituents selected from methyl and ethyl, CH═CH, (CH₂)ₙS or (CH₂)ₙO where n is 1, 2 or 3; and
V is CH, and
W is N, X is CH and Y is C,
W is N, X is N and Y is C,
W is C, X is N and Y is N, or
W is C, X is CH and Y is N; or
V is N, and
W is N, X is CH and Y is C,
W is N, X is N and Y is C, or
W is C, X is N and Y is N;
with the proviso that when V is CH, W is C, X is CH and Y is N, R² is CR⁶R⁷R⁸ as hereinbefore defined.

In one aspect the aryl group R¹ may be phenyl or naphthyl. Preferably, R¹ is phenyl optionally substituted by halogen, C₍₁₋₆₎alkyl, trifluoromethyl, C₍₁₋₆₎alkoxy, preferably, from 1 to 3 fluoro, more preferably, 4-fluoro or 2,3-difluoro.

In another aspect when W is C or N, R² may be methyl, ethyl, n-propyl, hydroxymethyl, hydroxyethyl, aminoethyl, dimethylaminomethyl, acetylaminoethyl, 2-(methoxyacetamido)ethyl, mesylaminoethyl, methanesulfonamidoethyl, (methoxyacetamido)ethyl, iso-propylcarboxymethyl, pyrimid-5-ylmethyl (optionally substituted by 2-methoxy, 2-trifluoromethyl, 2-(4-morpholino) or 2-dimethylamino), 2-oxo-pyrimid-5-ylmethyl or 1-methylpyrazol-4-ylmethyl. Preferably, R² is methyl, ethyl or 1-methylpyrazol-4-ylmethyl.

In another aspect when W is C, R² may be chloro, bromo, methoxy, methylthio, methylsulphinyl or ethylcarboxy.

In another aspect R³ may be hydrogen, methyl, 2-(diethylamino)ethyl, 2-(piperidin-1-yl)ethyl, 2-(pyrrolidin-1-yl)ethyl, 1-methyl-piperidin-4-yl, 1-ethyl-piperidin-4-yl, 1-ethyl-pyrrolidin-2-ylmethyl or 1-(2-methoxyethyl)piperidin-4-yl. Preferably R³ is 1-ethyl-piperidin-4-yl or 1-(2-methoxyethyl)piperidin-4-yl.

In another aspect R⁴ may be phenyl or pyridyl. Preferably, R⁴ is phenyl.

In another aspect R⁵ may be phenyl optionally substituted by halogen, or trifluoromethyl, preferably at the 4-position, or ethyl. Preferably, R⁵ is phenyl substituted by trifluoromethyl at the 4-position.

Preferably, R⁴ and R⁵ together form a 4-(phenyl)phenyl or a 2-(phenyl)pyridinyl substituent in which the remote phenyl ring may be optionally substituted by halogen or trifluoromethyl, preferably at the 4-position.

In another aspect R⁶ and R⁷ are hydrogen.

In another aspect R⁸ when an aryl group may be phenyl or naphthyl.

In another aspect R⁸ when a heteroaryl group may be a 5- or 6-membered, monocyclic heteroaryl group comprising 1 or 2 nitrogen heteroatoms.

Preferably, R⁸ is pyrimidyl optionally substituted by 1 or 2 substituents preferably selected from oxo, arylC₍₁₋₄₎alkyl (e.g. benzyl), C₍₁₋₆₎alkyl (e.g. methyl or ethyl), C₍₃₋₆₎cycloalkyl, hydroxy, C₍₁₋₄₎alkoxy (e.g. methoxy), carboxyC₍₁₋₆₎alkyl, C₍₁₋₆₎alkylcarboxyC₍₁₋₆₎alkyl, di-C₍₁₋₆₎alkylamino, and morpholino; or pyrazolyl optionally substituted by C₍₁₋₆₎alkyl (e.g. methyl or ethyl).

Compounds of the invention include:
N-(1-Ethylpiperidin-4-yl)-2-(6-(4-fluorobenzylthio)-3-methyl-4-oxo-4H-pyridazin-1-yl)-N-(4-(4-trifluoromethylphenyl)benzyl)acetamide bitartrate;
N-(1-(2-methoxyethyl)piperidinyl)-2-(1-ethyl-4-(4-fluorobenzylthio)-6-oxo-1,6-dihydro-pyridazin-3-yl)-N-(4-(4-trifluoromethylphenyl)benzyl)acetamide bitartrate;
N-(1-(2-methoxyethyl)piperidinyl)-2-(1-(1-methyl-4-pyrazolylmethyl)-4-(2-(2,3-difluorophenyl)ethyl)-6-oxo-1,6-dihydropyridazin-3-yl)-N-(4-(4-trifluoromethylphenyl)-benzyl)acetamide bitartrate; and
N(1-(2-methoxyethyl)piperidinyl)-2-(1-(1-methyl-4-pyrazolylmethyl)-4-(2,3-difluorobenzylthio)-6-oxo-1,6-dihydropyridazin-3-yl)-N-(4-(4-tfluoromethylphenyl)benzyl)acetamide bitartrate.

It will be appreciated that compounds of the present invention may comprise one or more chiral centres so that stereoisomers may be formed. The present invention encompasses all stereoisomers of the compounds of formula (I) including geometric isomers and optical isomers (eg. diastereoisomers and enantiomers) whether as individual stereoisomers isolated such as to be substantially free of the other stereoisomers (ie. pure) or as mixtures thereof including racemic modifications. An individual stereoisomer isolated such as to be substantially free of other stereoisomer (ie. pure) will preferably be isolated such that less than 10% preferably less than 1% especially less than 0.1% of the other stereoisomers is present.

Certain compounds of formula (I) may exist in one of several tautomeric forms. It will be understood that the present invention encompasses all tautomers of the compounds of formula (I) whether as individual tautomers or as mixtures thereof.

It will be appreciated that in some instances, compounds of the present invention may include a basic function such as an amino group as a substituent. Such basic functions may be used to form acid addition salts, in particular pharmaceutically acceptable salts. Pharmaceutically acceptable salts include those described by Berge, Bighley, and Monkhouse, J. Pharm. Sci., 1977, 66, 1-19. Such salts may be formed from inorganic and organic acids. Representative examples thereof include maleic, fumaric, benzoic, ascorbic, pamoic, succinic, bismethylenesalicylic, methanesulfonic, ethanedisulfonic, acetic, propionic, tartaric, salicylic, citric, gluconic, aspartic, stearic, palmitic, itaconic, glycolic, p-aminobenzoic, glutamic, taurocholic acid, benzenesulfonic, p-toluenesulfonic, hydrochloric, hydrobromic, sulfuric, cyclohexylsulfamic, phosphoric and nitric acids.

It will be appreciated that in some instances, compounds of the present invention may include a carboxy group as a substituent. Such carboxy groups may be used to form salts, in particular pharmaceutically acceptable salts. Pharmaceutically acceptable salts include those described by Berge, Bighley, and Monkhouse, J. Pharm Sci., 1977, 66, 1-19. Preferred salts include alkali metal salts such as the sodium and potassium salts.

When used herein, the term “alkyl” and similar terms such as “alkoxy” includes all straight chain and branched isomers. Representative examples thereof include methyl, ethyl, n-propyl, iso-propyl, n-butyl, sec-butyl, iso-butyl, t-butyl, n-pentyl and n-hexyl.

When used herein, the term “aryl” refers to, unless otherwise defined, a mono- or bicyclic aromatic ring system containing up to 10 carbon atoms in the ring system, for instance phenyl or naphthyl.

When used herein, the term “heteroaryl” refers to a mono- or bicyclic heteroaromatic ring system comprising up to four, preferably 1 or 2, heteroatoms each selected from oxygen, nitrogen and sulphur. Each ring may have from 4 to 7, preferably 5 or 6, ring atoms. A bicyclic heteroaromatic ring system may include a carbocyclic ring.

When used herein, the terms “halogen” and “halo” include fluorine, chlorine, bromine and iodine and fluoro, chloro, bromo and iodo, respectively.

It is to be understood that the present invention covers all combinations of substituent groups referred to above.

Since the compounds of the present invention, in particular compounds of formula (I), are intended for use in pharmaceutical compositions, it will be understood that they are each provided in substantially pure form, for example at least 50% pure, more suitably at least 75% pure and preferably at least 95% pure (% are on a wt/wt basis). Impure preparations of the compounds of formula (I) may be used for preparing the more pure forms used in the pharmaceutical compositions. Although the purity of intermediate compounds of the present invention is less critical, it will be readily understood that the substantially pure form is preferred as for the compounds of formula (I). Preferably, whenever possible, the compounds of the present invention are obtained in crystalline form.

When some of the compounds of this invention are allowed to crystallise or are re-crystallised from organic solvents, solvent of crystallisation may be present in the crystalline product. This invention includes within its scope such solvates. Similarly, some of the compounds of this invention may be crystallised or re-crystallised from solvents containing water. In such cases water of hydration may be formed. This invention includes within its scope stoichiometric hydrates as well as compounds containing variable amounts of water that may be produced by processes such as lyophilisation. In addition, different crystallisation conditions may lead to the formation of different polymorphic forms of crystalline products. This invention includes within its scope all polymorphic forms of the compounds of formula (I).

Compounds of the present invention are inhibitors of the enzyme lipoprotein associated phospholipase A₂ (Lp-PLA₂) and as such are expected to be of use in therapy, in particular in the treatment of atherosclerosis. In a further aspect therefore the present invention provides a compound of formula (I) for use in therapy.

The compounds of formula (I) are inhibitors of lysophosphatidylcholine production by Lp-PLA₂ and may therefore also have a general application in any disorder that involves endothelial dysfunction, for example atherosclerosis, diabetes, hypertension, angina pectoris and after ischaemia and reperfusion. In addition, compounds of formula (I) may have a general application in any disorder that involves lipid oxidation in conjunction with enzyme activity, for example in addition to conditions such as atherosclerosis and diabetes, other conditions such as rheumatoid arthritis, stroke, inflammatory conditions of the brain such as Alzheimer's Disease, myocardial infarction, reperfusion injury, sepsis, and acute and chronic inflammation.

Further applications include any disorder that involves activated monocytes, macrophages or lymphocytes, as all of these cell types express Lp-PLA₂. Examples of such disorders include psoriasis.

Accordingly, in a further aspect, the present invention provides for a method of treating a disease state associated with activity of the enzyme Lp-PLA₂ which method involves treating a patient in need thereof with a therapeutically effective amount of an inhibitor of the enzyme. The disease state may be associated with the increased involvement of monocytes, macrophages or lymphocytes; with the formation of lysophosphatidylcholine and oxidised free fatty acids; with lipid oxidation in conjunction with Lp PLA₂ activity; with ischemia and reperfusion; or with endothelial dysfunction.

Compounds of the present invention may also be of use in treating the above mentioned disease states in combination with an anti-hyperlipidaemic, anti-atherosclerotic, anti-diabetic, anti-anginal, anti-inflammatory, or anti-hypertension agent or an agent for lowering Lp(a). Examples of the above include cholesterol synthesis inhibitors such as statins, anti-oxidants such as probucol, insulin sensitisers, calcium channel antagonists, and anti-inflammatory drugs such as NSAIDs. Examples of agents for lowering Lp(a) include the aminophosphonates described in WO 97/02037, WO 98/28310, WO 98/28311 and WO 98/28312 (Symphar SA and SmnithKline Beecham).

A preferred combination therapy will be the use of a compound of the present invention and a statin. The statins are a well known class of cholesterol lowering agents and include atorvastatin, simvarstatin, pravastatin, cerivastatin, fluvastatin, lovastatin and ZD 4522 (also referred to as S-4522, rosuvastatin, Astra Zeneca). The two agents may be administered at substantially the same time or at different times, according to the discretion of the physician.

A further preferred combination therapy will be the use of a compound of the present invention and an anti-diabetic agent or an insulin sensitiser, as coronary heart disease is a major cause of death for diabetics. Within this class, preferred compounds for use with a compound of the present invention include the PPARgamma activators, for instance GI262570 (GlaxoSmithKline) and the glitazone class of compounds such as rosiglitazone (Avandia, GlaxoSmithKline), troglitazone and pioglitazone.

In therapeutic use, the compounds of the present invention are usually administered in a standard pharmaceutical composition. The present invention therefore provides, in a further aspect, a pharmaceutical composition comprising a compound of formula (I) and a pharmaceutically acceptable carrier, optionally with one or more other therapeutic compounds such as a statin or an anti-diabetic.

Suitable pharmaceutical compositions include those which are adapted for oral or parenteral administration or as a suppository, particularly for oral administration.

Compounds of formula (I) which are active when given orally can be formulated as liquids, for example syrups, suspensions or emulsions, tablets, capsules and lozenges. A liquid formulation will generally consist of a suspension or solution of the compound or pharmaceutically acceptable salt in a suitable liquid carrier(s) for example, ethanol, glycerine, non-aqueous solvent, for example polyethylene glycol, oils, or water with a suspending agent, preservative, flavouring or colouring agent. A composition in the form of a tablet can be prepared using any suitable pharmaceutical carrier(s) routinely used for preparing solid formulations. Examples of such carriers include magnesium stearate, starch, lactose, sucrose and cellulose. A composition in the form of a capsule can be prepared using routine encapsulation procedures. For example, pellets containing the active ingredient can be prepared using standard carriers and then filled into a hard gelatin capsule; alternatively, a dispersion or suspension can be prepared using any suitable pharmaceutical carrier(s), for example aqueous gums, celluloses, silicates or oils and the dispersion or suspension then filled into a soft gelatin capsule. Typical parenteral compositions consist of a solution or suspension of the compound of formula (I) in a sterile aqueous carrier or parenterally acceptable oil, for example polyethylene glycol, polyvinyl pyrrolidone, lecithin, arachis oil or sesame oil. Alternatively, the solution can be lyophilised and then reconstituted with a suitable solvent just prior to administration. A typical suppository formulation comprises a compound of formula (I) which is active when administered in this way, with a binding and/or lubricating agent such as polymeric glycols, gelatins or cocoa butter or other low melting vegetable or synthetic waxes or fats.

Preferably the composition is in unit dose form such as a tablet or capsule. Each dosage unit for oral administration contains preferably from 1 to 500 mg (and for parenteral administration contains preferably from 0.1 to 25 mg) of a compound of the formula (I). The daily dosage regimen for an adult patient may be, for example, an oral dose of between 1 mg and 1000 mg, preferably between 1 mg and 500 mg, or an intravenous, subcutaneous, or intramuscular dose of between 0.1 mg and 100 mg, preferably between 0.1 mg and 25 mg, of the compound of the formula (I), the compound being administered 1 to 4 times per day. Suitably the compounds will be administered for a period of continuous therapy, for example for a week or more.

According to a first process A, a compound of formula (I) may be prepared by reacting an acid compound of formula (II): 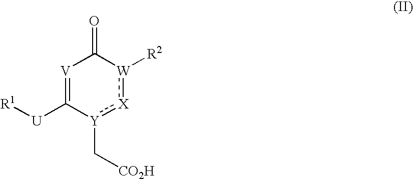
in which U, V, W, X, Y, R¹ and R² are as hereinbefore defined,
with an amine compound of formula (III):
R⁵—R⁴—CH₂NHR³ (III)
in which R³, R⁴ and R⁵ are as hereinbefore defined; under amide forming conditions.

Suitable amide forming conditions are well known in the art and include treating the acid of formula (II) with the amine of formula (III) in the presence of a coupling agent such as 1-(3-dimethyl-aminopropyl)-3-ethylcarbodiimide (DEC) or O-(7-azabenzotriazol-1-yl)-N,N,N′,N′-tetramethyluronium hexafluorophosphate (HATU) in an aprotic solvent such as dichloromethane or dimethylformamide (DMF).

A compound of formula (II) may be readily prepared from a corresponding ester of formula (IV): 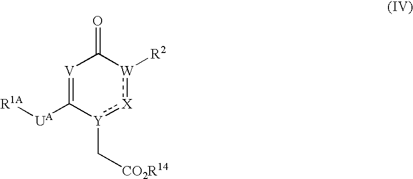
in which V, W, X, Y and R² are as hereinbefore defined, R^{1A} and U^{A} are R¹ and U as hereinbefore defined or a group, or groups, convertible to R¹ and U, and R¹⁴ is optionally substituted C₍₁₋₆₎alkyl, for example methyl, ethyl, t-butyl or 1,1-diphenylmethyl, by treating with a de-esterifying agent, for instance, for t-butyl, trifluoroacetic acid.

The ester of formula (IV) may be readily prepared by adapting standard pyridone, pyridazone and triazinone syntheses. General methods for preparing pyridones, pyridazones and triazinones are well known in the art and are described in, for example, Comprehensive Heterocyclic Chemistry, eds. A. R. Katritzky and C. W. Rees (Pergamon Press, Oxford 1984) and Comprehensive Heterocyclic Chemistry II, eds. A. R. Katritzky, C. W. Rees and E. F. V. Scriven (Pergamon Press, Oxford 1996).

For example, for compounds of formula (I) comprising a 6-oxo-1,6 dihydropyridazine ring, the ring may be prepared by reaction of a compound of formula (V) 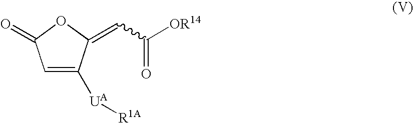
in which R^{1A} and U^{A} are R¹ and U as hereinbefore defined or a group, or groups, convertible to R¹ and U, and R¹⁴ is 1,1-diphenylmethyl,
with hydrazine hydrate in ethanol.

For compounds of formula (I) comprising a 4-oxo-4H-pyridazine ring, the ring may be prepared by reacting a compound of formula (VI) 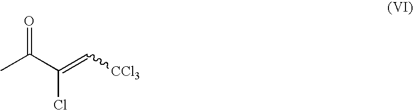
with a base such as DMF and a solution of semicarbazide hydrochloride in water and DMF, and subsequently forming the oxo group by treating with sodium in methanol and then refluxing with concentrated hydrochloric acid.

Conversion of R^{1A} and U^{A} to R¹ and U typically arises if a protecting group, or a group which can take part in subsequent reactions such as coupling reactions, is needed during the above reactions or during the preparation of the reactants. The conversion of R^{1A} and U^{A} to R¹ and U may be carried out at different stages in the synthesis of the compounds of formula (I) depending on the nature of R¹ and U, including as a final step.

R^{1A} and U^{A} may, for example, be a single group such as halo, for example chloro, bromo or iodo, which can be converted to give R¹ and U using one of the general methods for functional group transformation described in the literature provided that the method chosen is compatible with the other functional groups in the molecule. Functional group transformations are well known in the art and are described in for instance Comprehensive Organic Functional Group Transformations, eds. A. R. Katritzky, O. Meth-Cohn and C. W. Rees (Elsevier Science Ltd., Oxford, 1995), Comprehensive Organic Chemistry, eds. D. Barton and W. D. Ollis (Pergamon Press, Oxford, 1979), and Comprehensive Organic Transformations, R. C. Larock (VCH Publishers Inc., New York, 1989).

Thus, according to a further process B, compounds of formula (I) may be prepared by converting a compound of formula (VII): 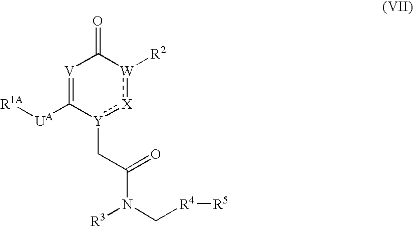
in which R^{1A}, U^{A}, V, W, Y, R², R³, R⁴ and R⁵ are as defined, to a compound to formula (I) by deprotection or functional group transformation.

The present invention will now be illustrated by the following examples.

### EXAMPLES

The structure and purity of the intermediates and examples was confirmed by 1H-NMR and (in nearly all cases) mass spectroscopy, even where not explicitly indicated below.
Intermediate A1—5,5,5-Trichloro-pent-3-ene-2-one

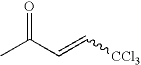

To a solution of diethyl(2-oxopropyl)phosphonate (20 g) in dry dimethylformamide (DMF) (400 ml) was cooled to 5° C. under argon. Sodium hydride (60% in oil, 4.53 g) was added portionwise over 1 h whilst maintaining the temperature between 5-8° C. The pale orange solution was stirred in an ice-bath and chloral (11.04 ml) in dry dimethoxyethane (200 ml) added over 1 h whilst keeping the temperature <8° C. After stirring in an ice bath for a further 30 min, the mixture was poured into water and extracted with diethyl ether (×3). The organic layers were combined, washed with brine, dried over MgSO₄ and evaporated under reduced pressure. The dark brown oil so formed was chromatographed on silica gel eluting with 9:1 hexane:ethyl acetate to give the title compound. ¹H NMR (CDCl₃) δ 2.38 (3H, s), 6.6 (1H, d), 7.04 (1H, d).
Intermediate A2—3,5,5,5-Tetrachloro-pent-3-ene-2-one

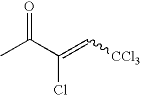

A solution of intermediate A1 (12.5 g) in carbon tetrachloride (75 ml) was cooled to 3° C. under argon. Iodine (1.2 g) was added followed by a solution of chlorine (4.15 g) in carbon tetrachloride (50 ml) over 1 h keeping the temperature below 5° C. The mixture was stirred in an ice bath for 30 min and washed with 10% aq sodium sulphite (75 ml). The aqueous layer was extracted with dichloromethane, the organic layers combined and washed with brine. This solution was dried over MgSO₄ and evaporated under reduced pressure to an oil that was dissolved in glacial acetic acid (40 ml) and anhydrous sodium acetate (5.47 g) added. The suspension was heated at 80° C. under argon for 2.5 h, cooled and evaporated under reduced pressure. The residue was partitioned between diethyl ether and water. The aqueous layer was washed with further diethyl ether and the combined organic layers washed with dilute brine, dried over MgSO₄ and evaporated under reduced pressure to a dark oil that was purified by chromatography on silica gel eluting with hexane. This gave the title compound (5.8 g). ¹H NMR (CDCl₃) δ 2.52 (3H, s), 7.56 (1H, s).
Intermediate A3—4,6-Dichloro-3-methylpyridazine.

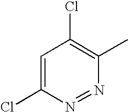

A solution of intermediate A2 (5.8 g) in DMP (30 ml) was cooled in an ice-bath and a solution of semicarbazide hydrochloride (2.93 g) in water (4.1 ml)/DMF (11.1 ml) added over 30 min. The cooling bath was removed, the mixture stirred at room temperature for 3 h and reduced to half volume under reduced pressure. Water was added, the mixture extracted with diethyl ether (×3), the combined organic layers washed with brine and dried over MgSO₄. The solvent was removed under reduced pressure and purified by chromatography on silica gel using hexane:ethyl acetate as eluent. This gave the title compound as a waxy solid (2.75 g). ¹H NMR (CDCl₃) δ 2.78 (3H, s), 7.54 (1H, s).
Intermediate A4—6-Chloro-4-methoxy-3-methylpyridazine.

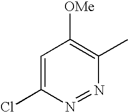

A solution of intermediate A3 (2.6 g) in dry tetrahydrofuran (45 ml) was cooled to 5° C. and a solution of sodium (0.405 g) in methanol (8.6 ml) added over 20 min. After a further 20 min in an ice bath and 15 min at room temperature, the mixture was evaporated to dryness and partitioned between water and ethyl acetate. The aqueous layer was washed with further ethyl acetate and the combined organic layers washed with brine, dried over MgSO₄ and evaporated under reduced pressure. The residue was triturated with diethyl ether/hexane and the light brown title compound (1.69 g) collected by filtration. ¹H NMR (CDCl₃) δ 2.56 (3H, s), 3.92 (3H, s), 6.81 (1H, s).
Intermediate A5—6-(4-Fluorobenzylthio)-4-methoxy-3-methylpyridazine.

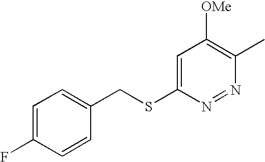

A solution of 4-fluorobenzylmercaptan (1.7 g) in dry tetrahydrofuran (THF) was added to a suspension of sodium hydride (60% in oil, 0.5 g) in dry THF (20 ml) over 15 min under argon. After 15 min at room temperature, a solution of intermediate A4 (1.6 g) in dry THF (20 ml) was added and the mixture was heated to reflux for 2 h. After cooling, the mixture was partitioned between ethyl acetate and dilute brine. The aqueous layer was extracted with further ethyl acetate and the combined organic layers were dried over MgSO₄ and evaporated under reduced pressure to give an oil that was chromatographed on silica gel using hexane:ethyl acetate as eluents. This gave the title compound (2.39 g). ¹H NMR (CDCl₃) δ 2.51 (3H, s), 3.83 (3H, s), 4.55 (2H, s), 6.56 (1H, s), 6.9-7.05 (2H, m), 7.35-7.5 (2H, m).
Intermediate A6—6-(4-Fluorobenzylthio)-3-methyl-1H-pyridazin-4-one.

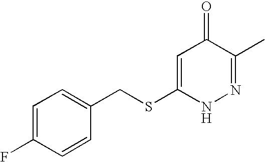

A mixture of intermediate A5 and conc.hydrochloric acid (20 ml) was stirred at reflux for 44 h. After cooling, the suspension was filtered and washed well with water and diethyl ether. The residue was suspended in saturated sodium bicarbonate, stirred well and filtered. The solid was washed with water and dried to give the title compound (1.8 g). The combined aqueous filtrates were washed with dichloromethane and the organic layer washed with dilute brine and dried over MgSO₄. The solution was evaporated under reduced pressure and triturated with diethyl ether to give further title compound (0.35 g). ¹H NMR (d₆-DMSO) δ 2.50 (3H, s), 4.37 (2H, s), 6.32 (1H, br s), 7.05-7.25 (2H, m), 7.35-7.5 (2H, m).
Intermediate A7—Ethyl(6-(4-fluorobenzylthio)-3-methyl-4-oxo-pyridazin-1-yl)acetate

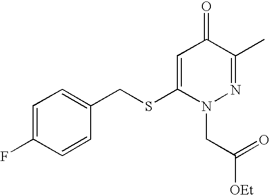

Intermediate A6 (0.5 g) was added to a suspension of sodium hydride (60% in oil, 0.088 g) in THF under argon. After a few minutes, DMF (5 ml) was added and the suspension stirred for a further 15 min. Ethyl bromoacetate (0.244 ml) was added. After a further 30 min further DMF (5 ml) was added and the mixture stirred at room temperature for a further 21 h. The solvent was removed under reduced pressure and the residue partitioned between ethyl acetate and brine. The aqueous layer was extracted with further ethyl acetate and the combined organic layers were combined, dried over MgSO₄ and evaporated under reduced pressure. The material so formed was chromatographed on silica gel using ethyl acetate and ethyl acetate:ethanol as eluents to give the title compound (0.12 g) ¹H NMR (CDCl₃) δ 1.29 (3H, t), 2.27 (3H, s), 4.15 (2H, s), 4.26 (2H, q), 4.84 (2H, s), 6.40 (1H, s), 6.95-7.1 (2H, m), 7.2-7.35 (2H, m); MS (APCI+) found (M+1)=337; C₁₆H₁₇FO₃N₂S requires 336.
Intermediate A8—(6-(4-fluorobenzylthio)-3-methyl-4-oxo-4H-pyridazin-1-yl)acetic acid.

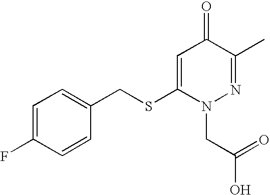

A solution of intermediate A7 (0.10 g) in dioxan (2 ml) was treated with a solution of sodium hydroxide (0.0119 g) in water (2 ml) and stirred at room temperature for 1 h. The solvent was removed under reduced pressure and the residue acidified to pH4 with 2M hydrochloric acid. The precipitate was collected, washed wit water and dried to give the title compound (0.39 g). ¹H NMR (d₆-DMSO) δ 2.09 (3H, s), 4.40 (2H, s), 4.89 (2H, s), 6.41 (1H, s), 7.05-7.25 (2H, m), 7.35-7.5 (2H, m).
Intermediate B1—1,1-Diphenylmethyl(3-bromo-5-oxo-5H-furan-2-ylidene)acetate.

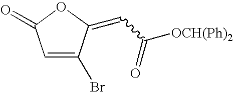

To a suspension of 1,1-Diphenylmethyl(3-bromo-5-oxo-5H-furan-2-ylidene)acetic acid (4.0 g) (see J. Org. Chem. 1994, 59(14), 4001-4003 and Tetrahedron Lett. 1988, 29(48), 6203-6206) in dichloromethane (100 ml) was added diphenyldiazomethane (4.26 g) portionwise. After stirring at room temperature for 18 h, the solution was concentrated to 25% volume and chromatographed on silica gel using dichloromethane:hexane as eluents. This gave the title compound (4.14 g). ¹H NMR (CDCl₃) δ 5.87 (1H, s), 6.64 (1H, s), 7.01 (1H, s), 7.2-7.5 (10H, m).
Intermediate B2—1,1-Diphenylmethyl(4-bromo-6-oxo-1,6-dihydropyridazin-3-yl)acetate

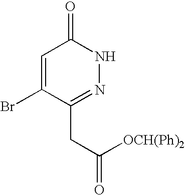

To a suspension of intermediate B1 (4.06 g) in ethanol (40 ml) at room temperature was added hydrazine hydrate (0.51 ml). After 10 min the mixture was heated to reflux for 18 h, cooled and the solid so formed filtered and washed with ethanol and diethyl ether to give the title compound (3.63 g). ¹HNMR (CDCl₃) δ 3.92 (2H, s), 6.94 (1H, s), 7.2-7.4 (11H, m), 11.15 (1H, br s).
Intermediate B3—1,1-Diphenylmethyl(4-bromo-1-ethyl-6-oxo-1,6-dihydropyridazin-3-yl)acetate.

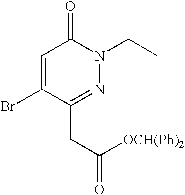

A mixture of intermediate B2 (0.2 g) in dry DMF at 40° C. was treated with sodium hydride (0.022 g) under argon. After 10 min ethyl iodide (0.044 ml) was added and stirred at room temperature. After 2 h, the solvent was removed under reduced pressure and the residue partitioned between dichloromethane and saturated sodium metabisulphite. The organic layer was washed with brine, dried over MgSO₄ and evaporated under reduced pressure. The residue was chromatographed on silica gel eluting with dichloromethane:hexane. This gave the title compound (0.181 g). ¹H NMR (CDCl₃) δ 1.31 (3H, t), 3.91 (2H, s), 4.12 (2H, q), 6.94 (1H, s), 7.15-7.4 (11H, m).
Intermediate B4—Methyl(1-ethyl-4-(4-fluorobenzylthio)-6-oxo-1,6-dihydropyridazin-3-yl)acetate

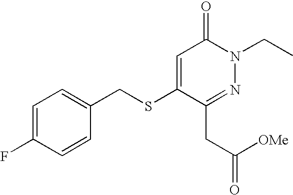

A solution of intermediate B3 (0.079 g) in methanol was added to a solution of sodium 4-fluorobenzylthiolate (from sodium (0.0043 g) in methanol (1 ml). After 15 min the solvent was removed under reduce pressure and the residue chromatographed on silica gel using dichloromethane:hexane as eluents. This gave the title compound contaminated with its biphenylmethyl ester (0.14 g). ¹HNMR (CDCl₃) δ 1.3 (3H, t), 3.68 (2H, s), 3.73 (3H, s), 4.0-4.2 (4H, m), 6.60 (1H, s), 6.95-7.1 (2H, m), 7.2-7.35 (2H, m); MS (APCI+) found (M+1)=337; C₁₆H₁₇FN₂O₃S requires 336.

Similarly prepared was:
Intermediate B11—1,1-Diphenylmethyl(1-(1-methylpyrazol-4-ylmethyl)-4-(4-fluorobenzylthio)-6-oxo-1,6-dihydropyridazin-3-yl)acetate and Methyl(1-(1-methylpyrazol-4-ylmethyl)-4-(4-fluorobenzylthio)-6-oxo-1,6-dihydropyridazin-3-yl)acetate

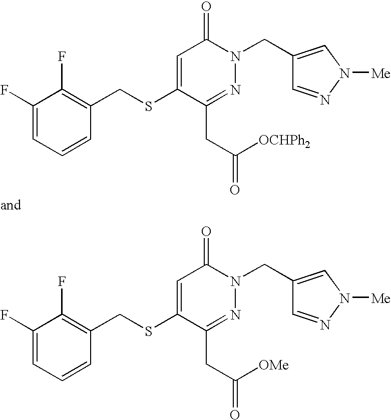

From intermediate B8 and 2,3-difluorobenzylthiol.
Intermediate B5—(1-Ethyl-4-(4-fluorobenzylthio)-6-oxo-1,6-dihydropyridazin-3-yl)acetic acid

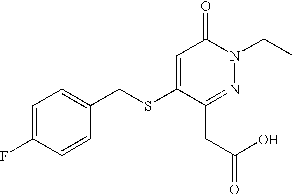

To the mixture of esters containing B4 above (0.252 g) in methanol (3 ml) was added a solution of sodium hydroxide (0.5M, 1.03 ml) and the suspension stirred for 24 h. The mixture was heated to reflux for 10 min and 2 drops of sodium hydroxide (0.5M) added. After heating at reflux for a further 15 min, the mixture was cooled, acidified with dilute hydrochloric acid and the solvent removed under reduced pressure. The residue was partitioned between dichloromethane and water and the organic layer washed with brine and dried over MgSO₄. Removal of the solvent under reduced pressure and trituration of the product with diethyl ether gave the title compound (0.118 g). ¹HNMR (CDCl₃) δ 1.33 (3H, t), 3.70 (2H, s), 4.054.2 (4H, m), 6.86 (1H, s), 6.9-7.1 (2H, m), 7.25-7.4 (2H, m).

Similarly prepared was
Intermediate B12—(1-(1-Methylpyrazol-4-ylmethyl)-4-(4-fluorobenzylthio)-6-oxo-1,6-dihydropyridazin-3-yl)acetic acid

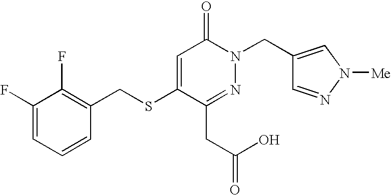

From intermediate B 11.
Intermediate B6—2,3-Difluorophenylacetylene

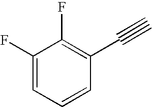

Trimethylsilylacetylene (4.38 ml) was added to a mixture of 1-bromo-2,3-difluorobenzene (4.99 g), copper(I) iodide (0.493 g), tetrakistriphenylphosphine palladium (1.49 g) and triethylamine (20 ml) under argon. The mixture was stirred and heated to reflux for 18 h. The solvent was removed under reduced pressure and the residue taken up in ethyl acetate and filtered through Celite. The filtrate was washed with saturated ammonium chloride and brine, dried over MgSO₄ and carefully evaporated under reduced pressure. Methanol was added and the mixture was carefully evaporated once more. The material so formed was dissolved in methanol (10 ml) and added to a solution of potassium hydroxide (14.5 g) in methanol (30 ml) with stirring. After 18 h, the solution was diluted with water and extracted with diethyl ether. The combined diethyl ether layers were dried over MgSO₄ and carefully evaporated and distilled (b.p. 65-115° C./33 mm Hg) to give the title compound. ¹H NMR (CDCl₃) δ 6.99-7.44 (all protons).
Intermediate B7—4-Bromomethyl-1-methylpyrazole hydrobromide

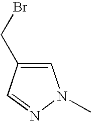

To a suspension of 4-formyl-1-methylpyrazole (6 g) in THF (30 ml) at 0° C. under argon was added lithium aluminium hydride (1M in TBF, 27.2 ml) dropwise. The mixture was allowed to warm to room temperature and stirred for 1.5 h. Water (1 ml), 10% sodium hydroxide (1 ml) and water (3 ml) were added sequentially with care, the mixture stirred for a further 30 min and evaporated under reduced pressure. The residue was partitioned between dichloromethane and brine, dried over MgSO₄ and evaporated under reduced pressure to give an oil (3.48 g). A portion of this material (1.15 g) in acetic acid (5 ml) was mixed with 48% hydrogen bromide in acetic acid and the mixture heated to reflux for 5 h. The solvent was removed under reduced pressure and the residue crystallised from dichloromethane and diethyl ether to give the title compound (1.6 g) ¹H NMR (D20) δ 4.09 (3H, s), 4.60 (2H, s), 8.07 (2H, s).
Intermediate B8-1,1-Diphenylmethyl(4-bromo-1-(1-methylpyrazol-4-ylmethyl)-6-oxo-1,6-dihydropyridazin-3-yl)acetate

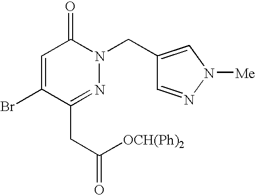

To a solution of intermediate B2 (0.5 g) in dry DMF (10 ml) was added sodium hydride (60% in oil, 0.05 g) at room temperature with stirring, under argon. The mixture was stirred at room temperature for 20 min and intermediate B7 (0.32 g) in dry DMF (3 ml) added followed by further sodium hydride (60% in oil, 0.055 g). The mixture was stirred at room temperature for 4.5 h and evaporated under reduced pressure. The residue was partitioned between dichloromethane and water and the organic layer washed with brine and dried over MgSO₄. Removal of the solvent under reduced pressure followed by chromatography on silica gel using ethyl acetate:dichloromethane as eluents gave the title compound (0.24 g). ¹H NMR (CDCl₃) δ 3.81 (3H, s), 3.91 (21, s), 5.07 (2H, s), 6.94 (1H, s), 7.19 (1H, s), 7.2-7.4 (10H, m), 7.42 (1H, s), 7.49 (1H, s).
Intermediate B9—1,1-Diphenylmethyl(4-(2,3-difluorophenylethynyl)-1-(1-methylpyrazol-4-ylmethyl)-6-oxo-1,6-dihydropyridazin-3-yl)acetate

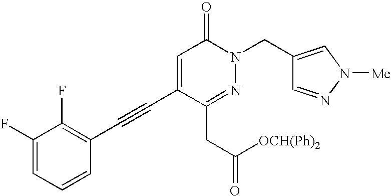

To a solution of intermediate B6 (0.2 g) in triethylamine (10 ml) was added intermediate B8 (0.44 g), bistriphenylphosphine palladium dichloride (0.031 g), copper(I) iodide (0.009 g) and dichloromethane (2 ml). The mixture was heated at 70° C. for 18 h, evaporated under reduced pressure and chromatographed on silica gel using dichloromethane:ethyl acetate as eluents. This gave the title compound (0.44 g). ¹H NMR (CDCl₃) δ 3.83 (3H, s), 3.95 (2H, s), 5.11 (2H, s), 6.85-7.05 (4H, m), 7.1-7.35 (111H, m), 7.4-7.6(2H, m); MS (APCI+) found (M+1)=551; C₃₂H₂₄F₂N₄O₃ requires 550.
Intermediate B10—(4-(2-(2,3-difluorophenyl)ethyl)-1-(1-methylpyrazol-4-ylmethyl)-6-oxo-1,6-dihydropyridazin-3-yl)acetic acid

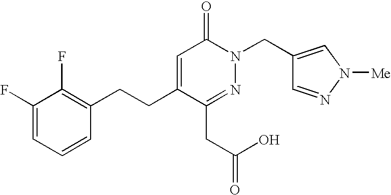

Intermediate B9 (0.44 g) was dissolved in DMF (10 ml) and 10% palladium on charcoal (0.2 g) added carefully. The mixture was hydrogenated at room temperature and pressure for 18 h, filtered through Celite and the filtrate evaporated under reduced pressure. The residue was partitioned between diethyl ether and 0.5M sodium hydroxide. The organic layer was washed with further sodium hydroxide and the combined aqueous layers washed with diethyl ether and acidified to pH1 with hydrochloric acid. The precipitate so formed was extracted with diethyl ether and the combined extracts dried over MgSO₄ and evaporated to give the title compound (0.19 g). ¹H NMR (46-DMSO) δ 2.65-2.8 (2H, m), 2.9-3.0 (2H, m), 3.67 (2H, s), 3.77 (3H, s), 5.01 (2H, s), 6.75 (1H, s), 7.1-7.2 (2H, m), 7.2-7.35 (1H, m), 7.36 (1H, s), 7.64 (1H, s).

The following amines are known in the literature.

| No. | Reference | Structure | Name |
|---|---|---|---|
| C1 | WO 01/60805 | 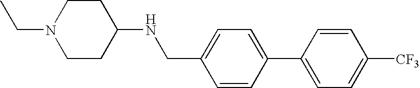 | N-(1-Ethylpiperidin-4-yl)-4-(4-tri- fluoromethyl- phenyl)benzylamine |
| C2 | WO 01/60805 | 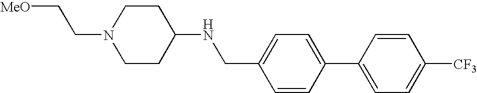 | N-(1-(2-meth- oxyethyl)piperidin-4-yl)-4-(4-tri- fluoromethyl- phenyl)benzylamine |

### Example 1

### N-(1-Ethylpiperidin-4-yl)-2-(6-(4-fluorobenzylthio)-3-methyl-4-oxo-4H-pyridazin-1-yl)-N-(4-(4-trifluoromethylphenyl)benzyl)acetamide bitartrate

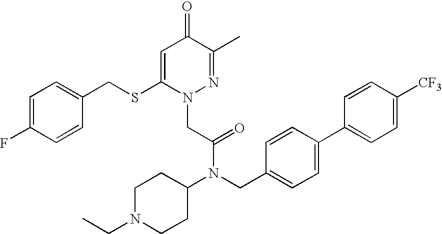

O-(7-Azabenzotriazol-1-yl)-N,N,N′,N′-tetramethyluronium hexafluorophosphate (HATU) (0.187 g, 0.6 mmol) was added to a mixture of intermediate A8 (0.114 g), amine C1 (0.09 g) and diisopropylethylamine (0.104 ml) in dimethylformamide (2 ml) and the resultant solution stirred for 2 h under argon. The solvent was evaporated and the residue diluted with dichloromethane (30 ml) and washed successively with saturated ammonium chloride, saturated sodium bicarbonate and dilute brine. The organic layer was dried (K₂CO₃) and the solvent evaporated. The residue was purified by flash chromatography (CH₂Cl₂/MeOH) to give the free base of the title compound (0.095 g). ¹H NMR (CDCl₃) δ 0.95 (3H, m), 1.6-2.1 (6H, m), 2.26+2.29 (3H, 2xs), 2.3-2.5 (2H, q), 2.9-3.1 (2H, m), 4.1+4.16 (2H, 2xs), 4.54.7 (3H, m), 4.82+5.09 (2H, 2xs), 6.42+6.45 (1H, 2xs), 6.85-7.1 (2H, m), 7.15-7.4 (4H, m), 7.47.55 (2H, m), 7.6-7.8 (4H, m); MS (APCI+) found (M+1)=653; C₃₅H₆F₄N₄O₂S requires 652.

The amine (0.09 g) was dissolved in methanol (2 ml) and tartaric acid (0.022 g) added. After sting for 15 min the solvent was evaporated and the residue triturated from diethyl ether to afford the title compound (0.10 g). ¹H NMR (d₆-DMSO) δ 0.9-1.15 (3H, m), 1.55-1.95 (4H, m), 2.09+2.12 (3H, 2xs), 2.2-2.7 (4H, m), 2.95-3.25 (2H, m), 3.754.0+4.2-4.35 (1H, 2xbr), 4.11 (2H, s), 4.34+4.31 (2H, 2xs), 4.58+4.69 (2H, 2xs), 5.01+5.37 (2H, 2xs), 6.43+6.44 (1H, 2xs), 6.95-7.25 (2H, m), 7.25-7.5 (4H, m), 7.5-7.75 (2H, m), 7.75-7.95 (4H, m).

The following examples were prepared by the method of Example 1.

| Example No. | Precursors | Structure | Name |
|---|---|---|---|
| 2 | B5, C2 | 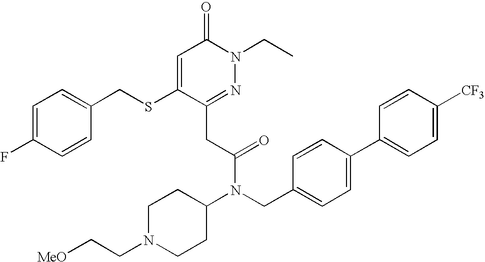 | N-(1-(2-methoxyethyl)-pipe- ridin-4-yl)-2-(1-ethyl-4-(4-fluoro- benzylthio)-6-oxo-1,6-di- hydropyridazin-3-yl)-N-(4-(4-tri- fluoromethyl-phe- nyl)benzyl)acetamide bitartrate |
| 3 | B10, C2 | 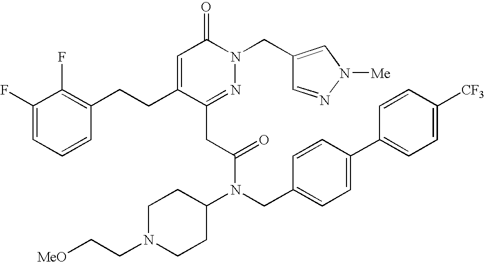 | N-(1-(2-methoxyethyl)-pipe- ridin-4-yl)-2-(1-(1-methyl-4-py- razolylmethyl)-4-(2-(2,3-di- fluorophenyl)ethyl)-6-oxo-1,6-di- hydropyridazin-3-yl)-N-(4-(4-tri- fluoromethylphenyl)-ben- zyl)acetamide bitartrate |
| 4 | B12, C2 | 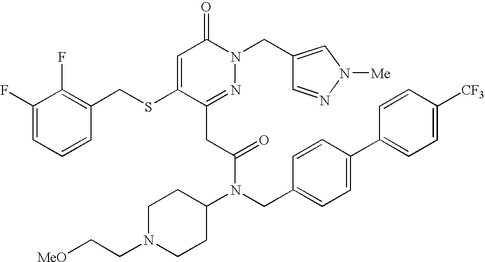 | N-(1-(2-methoxyethyl)-pipe- ridin-4-yl)-2-(1-(1-methyl-4-py- razolylmethyl)-4-(2,3-di- fluorobenzylthio)-6-oxo-1,6-di- hydropyridazin-3-yl)-N-(4-(4-tri- fluoromethylphenyl)-ben- zyl)acetamide bitartrate |

Biological Data
1. Screen for Lp-PLA₂ Inhibition.

Enzyme activity was determined by measuring the rate of turnover of the artificial substrate (A) at 37° C. in 5 mM HEPES (N-2-hydroxyethylpiperazine-N′-2-ethanesulphonic acid) buffer containing 150 mM NaCl, pH 7.4.

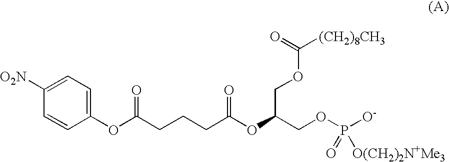

Assays were performed in 96 well titre plates.

Recombinant Lp-PLA₂ was purified to homogeneity from baculovirus infected Sf9 cells, using a zinc chelating column, blue sepharose affinity chromatography and an anion exchange column. Following purification and ultrafiltration, the enzyme was stored at 6 mg/ml at 4° C. Assay plates of compound or vehicle plus buffer were set up using automated robotics to a volume of 170 μl. The reaction was initiated by the addition of 20 μl of 10× substrate (A) to give a final substrate concentration of 20 μM and 10 μl of diluted enzyme to an approximate final 0.1 nM Lp-PLA₂.

The reaction was followed at 405 nm and 37° C. for 20 minutes using a plate reader with automatic mixing. The rate of reaction was measured as the rate of change of absorbance.

Results

The compounds described in the Examples were tested as described above and had IC₅₀ values in the range <0.1 to 100 nM.

1. A compound of formula (I): 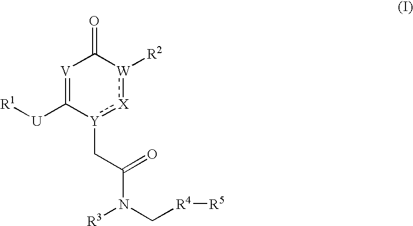
in which:
R¹ is an aryl group, optionally substituted by 1, 2, 3 or 4 substituents which may be the same or different selected from the group consisting of C₍₁₋₆₎alkyl, C₍₁₋₆₎alkoxy, C₍₁₋₆₎alkylthio, hydroxy, halogen, CN, mono to perfluoro-C₍₁₋₄₎alkyl, mono to perfluoro-C₍₁₋₄₎alkoxyaryl, and arylC₍₁₋₄₎alkyl;
when W is C, R² is hydrogen, halogen, C₍₁₋₆₎alkyl, C₍₁₋₃₎alkoxy, hydroxyC₍₁₋₃₎alkyl, C₍₁₋₃₎alkylthio, C₍₁₋₃₎alkylsulphinyl, aminoC₍₁₋₃₎alkyl, mono- or di-C₍₁₋₃₎alkylaminoC₍₁₋₃₎alkyl, C₍₁₋₃₎alkylcarbonylaminoC₍₁₋₃₎alkyl, C₍₁₋₃₎alkoxyC₍₁₋₃₎alkylcarbonylaminoC₍₁₋₃₎alkyl, C₍₁₋₃₎alkylsulphonylaminoC₍₁₋₃₎alkyl, C₍₁₋₃₎alkylcarboxy, or CR⁶R⁷R⁸; or
when W is N, R² is hydrogen, C₍₁₋₃₎alkyl, hydroxyC₍₁₋₃₎alkyl, aminoC₍₁₋₃₎alkyl, mono- or di-C₍₁₋₃₎alkylaminoC₍₁₋₃₎alkyl, C₍₁₋₃₎alkylcarbonylaminoC₍₁₋₃₎alkyl, C₍₁₋₃₎alkoxyC₍₁₋₃₎alkylcarbonylaminoC₍₁₋₃₎alkyl, C₍₁₋₃₎alkylsulphonylaminoC₍₁₋₃₎alkyl, or CR⁶R⁷R⁸;
R³ is hydrogen, C₍₁₋₆₎alkyl which may be unsubstituted or substituted by 1, 2 or 3 substituents selected from hydroxy, halogen, OR⁹, COR⁹, carboxy, COOR⁹, CONR¹⁰R¹¹, NR¹⁰R¹¹, NR⁹COR¹², mono- or di-(hydroxyC₍₁₋₆₎alkyl)amino and N-hydroxyC₍₁₋₆₎alkyl-N—C₍₁₋₆₎alkylamino; or
R³ is Het-C₍₀₋₄₎alkyl in which Het is a 5- to 7-membered heterocyclyl ring comprising N and optionally O or S, and in which N may be substituted by COR⁹, COOR⁹, CONR¹⁰R¹¹, or C₍₁₋₆₎alkyl optionally substituted by 1, 2 or 3 substituents selected from hydroxy, halogen, OR⁹, COR⁹, carboxy, COOR⁹, CONR¹⁰R¹¹ or NR¹⁰R¹¹;
R⁴ is an aryl or a heteroaryl ring optionally substituted by 1, 2, 3 or 4 substituents which may be the same or different selected from the group consisting of C₍₁₋₆₎alkyl, C₍₁₋₆₎alkoxy, C₍₁₋₆₎alkylthio, arylC₍₁₋₆₎alkoxy, hydroxy, halogen, CN, COR⁹, carboxy, COOR⁹, NR⁹COR¹², CONR¹⁰R¹¹, SO₂NR¹⁰R¹¹, NR⁹SO₂R¹², NR¹⁰R¹¹, mono to perfluoro-C₍₁₋₄₎alkyl and mono to perfluoro-C₍₁₋₄₎alkoxy;
R⁵ is an aryl or a heteroaryl ring which is further optionally substituted by 1, 2, 3 or 4 substituents which may be the same or different selected from the group consisting of C₍₁₋₁₈₎alkyl, C₍₁₋₁₈₎alkoxy, C₍₁₋₆₎alkylthio, C₍₁₋₆₎alkylsulfonyl, arylC₍₁₋₆₎alkoxy, hydroxy, halogen, CN, COR⁹, carboxy, COOR⁹, CONR¹⁰R¹¹, NR⁹COR¹², SO₂NR¹⁰R¹¹, NR⁹SO₂R¹², NR¹⁰R¹¹, mono to perfluoro-C₍₁₋₄₎alkyl and mono to perfluoro-C₍₁₋₄₎alkoxy, or C₍₅₋₁₀₎alkyl;
R⁶ and R⁷ are each hydrogen or C₍₁₋₄₎alkyl, or R⁶ and R⁷ together with the intervening carbon atom form a C₍₃₋₆₎cycloalkyl ring;
R⁸ is an aryl or heteroaryl group, optionally substituted by 1, 2, 3 or 4 substituents which may be the same or different selected from C₍₁₋₁₈₎alkyl, C₍₁₋₁₈₎alkoxy, C₍₁₋₁₈₎alkylthio, arylC₍₁₋₁₈₎alkoxy, hydroxy, halogen, CN, COR⁹, carboxy, COOR⁹, CONR¹⁰R¹¹, NR⁹COR¹², SO₂NR¹⁰R¹¹, NR⁹SO₂R¹², NR¹⁰R¹¹, mono to perfluoro-C₍₁₋₄₎alkyl and mono to perfluoro-C₍₁₋₄₎alkoxy; or
R⁸ is an aryl or heteroaryl group, optionally substituted by 1 substituent selected from CH₂COOH or a salt thereof, CH₂COOR¹³, CH₂CONR¹⁰R¹¹, CH₂CN, (CH₂)ₘNR¹⁰R¹¹, (CH₂)ₘOH and (CH₂)ₘOR⁹ where m is an integer from 1 to 3, optionally in combination with a further substituent selected from the group consisting of C₍₁₋₁₈₎alkyl, C₍₁₋₁₈₎alkoxy, C₍₁₋₁₈₎alkylthio, arylC₍₁₋₁₈₎alkoxy, hydroxy, halogen, CN, COR⁹, carboxy, COOR⁹, CONR¹⁰R¹¹, NR⁹COR¹², SO₂NR¹⁰R¹¹, NR⁹SO₂R¹², NR¹⁰R¹¹, mono to perfluoro-C₍₁₋₄₎alkyl and mono to perfluoro-C₍₁₋₄₎alkoxy;
R⁹ and R¹² are independently hydrogen or C₍₁₋₁₂₎alkyl;
R¹⁰ and R¹¹ which may be the same or different is each selected from hydrogen, or C₍₁₋₁₂₎alkyl, or R¹⁰ and R¹¹ together with the nitrogen to which they are attached form a 5- to 7 membered ring optionally containing one or more further heteroatoms selected from oxygen, nitrogen and sulphur, and optionally substituted by one or two substituents selected from hydroxy, oxo, C₍₁₋₄₎alkyl, C₍₁₋₄₎alkylcarboxy, aryl, e.g. phenyl, or aralkyl;
R¹³ is C₍₁₋₄₎alkyl or a pharmaceutically acceptable in vivo hydrolysable ester group;
U is a C₍₂₋₄₎alkylene group optionally substituted by 1, 2 or 3 substituents selected from methyl and ethyl, CH═CH, (CH₂)ₙS or (CH₂)ₙO where n is 1, 2 or 3; and
V is CH, and
W is N, X is CH and Y is C,
W is N, X is N and Y is C,
W is C, X is N and Y is N, or
W is C, X is CH and Y is N; or
V is N, and
W is N, X is CH and Y is C,
W is N, X is N and Y is C, or
W is C, X is N and Y is N;
and pharmaceutically acceptable salts thereof, with the proviso that when V is CH, W is C, X is CH and Y is N, R² is CR⁶R⁷R⁸ as hereinbefore defined.

2. A compound according to claim 1 wherein R¹ is phenyl optionally substituted by halogen, C₍₁₋₆₎ alkyl, trifluoromethyl or C₍₁₋₆₎ alkoxy.

3. A compound according to claim 1 wherein R³ may be hydrogen, methyl, 2-(diethylamino)ethyl, 2-(piperidin-1-yl)ethyl, 2-(pyrrolidin-1-yl)ethyl, 1-methyl-piperidin-4-yl, 1-ethyl-piperidin-4-yl, 1-ethylpyrrolidin-2-ylmethyl or 1-(2-methoxyethyl)piperidin-4-yl.

4. A compound according to claim 1 wherein R⁴ is phenyl or pyridyl.

5. A compound according to claim 1 wherein R⁵ is phenyl optionally substituted by halogen or trifluoromethyl.

6. A compound according to claim 1 wherein W is C or N and R² is methyl, ethyl, n-propyl, hydroxymethyl, hydroxyethyl, aminoethyl, dimethylaminomethyl, acetylaminoethyl, 2-(methoxyacetamido)ethyl, mesylaminoethyl, methanesulfonamidoethyl, (methoxyacetamido)ethyl, iso-propylcarboxymethyl, pyrimid-5-ylmethyl (optionally substituted by 2-methoxy, 2-trifluoromethyl, 2-(4-morpholino) or 2-dimethylamino), 2-oxo-pyrimid-5-ylmethyl or 1-methylpyrazol-4-ylmethyl.

7. A compound according to claim 1 which is
N-(1-Ethylpiperidin-4-yl)-2-(6-(4-fluorobenzylthio)-3-methyl-4-oxo-4H-pyridazin-1-yl)-N-(4-(4-trifluoromethylphenyl)benzyl)acetamide bitartrate;
N-(1-(2-methoxyethyl)piperidin-4-yl)-2-(1-ethyl-4-(4-fluorobenzylthio)-6-oxo-1,6-dihydropyridazin-3-yl)-N-(4-(4-trifluoromethylphenyl)benzyl)acetamide bitartrate;
N-(1-(2-methoxyethyl)piperidin-4-yl)-2-(1-(1-methyl-4-pyrazolylmethyl)-4-(2-(2,3-difluorophenyl)ethyl)-6-oxo-1,6-dihydropyridazin-3-yl)-N-(4-(4-trifluoromethylphenyl)benzyl)acetamide bitartrate; and
N-(1-(2-methoxyethyl)piperidin-4-yl)-2-(1-(1-methyl-4-pyrazolylmethyl)-4-(2,3-difluorobenzylthio)-6-oxo-1,6-dihydropyridazin-3-yl)-N-(4-(4-trifluoromethylphenyl)benzyl)acetamide bitartrate.

8. A pharmaceutical composition comprising a compound of formula (I) as claimed in claim 1 and a pharmaceutically acceptable carrier.

9. A compound of formula (I) as claimed in claim 1 for use treating atherosclerosis.

10. (Deleted)

11. A method of treating a disease state associated with activity of the enzyme Lp-PLA₂ which method involves treating a patient in need thereof with a therapeutically effective amount of a compound of formula (I) as claimed in claim 1.

12. A process for preparing a compound of formula (I) as defined in claim 1 which process comprises reacting an acid compound of formula (II): 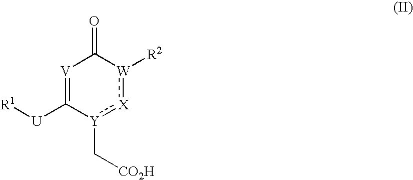
in which U, V, W, X, Y, R¹ and R² are as hereinbefore defined, with an amine compound of formula (III):
R⁵—R⁴—CH₂NHR³ (III)
in which R³, R⁴ and R⁵ are as hereinbefore defined; under amide forming conditions.
